(19) United States
(12) Patent Application Publication
(10) Pub. No.: US 2013/0172758 A1
(43) Pub. Date: Jul. 4, 2013

(54) Trans-septal Puncture Catheter Incorporating Intra-cardiac Echocardiography

(71) Applicant: Eduardo de Marchena, Miami, FL (US)

(72) Inventor: Eduardo de Marchena, Miami, FL (US)

(21) Appl. No.: 13/536,253

(22) Filed: Jun. 28, 2012

Related U.S. Application Data
(60) Provisional application No. 61/504,579, filed on Jul. 5, 2011.

(51) Int. Cl. A61B 8/00; A61B 8/08; A61B 17/34; A61B 8/12; A61B 18/14

(52) U.S. Cl. CPC A61B 8/445; A61B 8/12; A61B 18/1477; A61B 17/3478; A61B 8/4483; A61B 8/0883
USPC 600/464

(57) ABSTRACT

This invention relates to a pre-formed shape-memory trans-septal puncture catheter incorporating an intra cardiac echocardiography sensor and smart sheath and a related method of use

### CROSS REFERENCE TO RELATED APPLICATIONS

Not applicable.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

1. Field of the Invention

This invention relates to an ultrasonic and interventional catheter incorporating both an intra cardiac echocardiography sensor and a pre-formed shape-memory needle and needle sheath, “smart sheath”.

2. Background of the Invention

There is a need for cardiac doctors and surgeons to be able to performing an intra-cardiac transseptal puncture as a preliminary step in performing left-side cardiac catheterization therapies and diagnostic procedures. U.S. Pat. No. 7,637,870 discloses a catheter device for penetrating the sidewall of a blood vessel and associated tissue to gain access to adjacent blood vessel. The patent discloses an imaging device on the distal end of a catheter and a penetrating device that extends laterally from the catheter. The imaging device is formed into the distal end of the catheter and is disclosed to image only the location of the penetration for orientation purposes. Real-time imaging is not provided. The therapeutic device of the '870 patent is specifically disclosed to be a device for puncturing blood vessels. The device is not specifically disclosed to provide a puncture device for puncturing the heart septum. The imaging device of the '870 patent is located at the tip of the catheter and is not independently movable in relation to the catheter. The penetrator of the '870 patent is disposed within the catheter and is laterally extendable from the catheter. There is no co-axial, double catheter system whereby the needle/introducer is itself contained inside a catheter. The imaging device of the '870 patent is not movable and is only able to visualize where the penetrator will be deployed. The ability to visualize the actual needle/introducer deployment is not disclosed. Neither the penetrator nor the imaging device of the '870 patent are disclosed to be formed of shape-memory material.

U.S. Pat. No. 6,645,195 discloses a drug delivery device for delivering drugs to the body. The patent discloses the use of two catheters. The first catheter is disclosed to contain both an ultrasonic element and a drug delivery device in a parallel arrangement. The ultrasonic element is retractable within the first catheter while the drug delivery device is extendable beyond the distal end of the first catheter. A second catheter is thereafter disclosed to include a complementary ultrasonic element. The therapeutic device of the '195 patent is specifically disclosed to be either a drug delivery device, i.e., a needle. The device is not specifically disclosed to provide a puncture device for puncturing the heart septum. The ultrasonic imaging device of the '195 patent uses two imaging devices to provide for proper imaging. The invention does not require this and utilizes only one imaging device. The imaging device in the first (main) catheter of the '195 invention remains in the catheter and is used to position the catheter rather than observe the procedure, as in your invention. The '195 patent's imaging device and drug delivery device are arranged in parallel in the first catheter. This results in the use of a larger catheter whereas the co-axial arrangement of your invention provides for reduced catheter size. The '195 patent discloses the use of memory-shape materials in its guide wires, but does not disclose its use for the imaging device.

U.S. Pat. Nos. 5,345,940 & 5,325,860 disclose a catheter system for delivery of therapeutic devices. The patent discloses a catheter having a distally located built-in imaging device. Parallel to this imaging device, and still within the catheter, there is disposed a lumen for a guide wire and a lumen for a therapeutic device. The imaging device is disclosed to be oriented such that, in operation, the therapeutic device may be visualized. The imaging device, however, is not disclosed to be able to image the guide wire. The therapeutic device of the '940 & '860 patents is specifically disclosed to be either an ablation device, biopsy device, or implantation device. The device is not specifically disclosed to provide a puncture device for puncturing the heart septum. The ultrasonic imaging device of the '940 & '860 patents is formed into the catheter. The guide wire port of the '940 & '860 patents is not viewable by its imaging device. The guide wire lumen, therapeutic device lumen and imaging device instrumentation run parallel to one another in the '940 & '860 patents. This results in the use of a larger catheter whereas the co-axial arrangement of your invention provides for reduced catheter size.

### BRIEF SUMMARY OF THE INVENTION

The invention set forth herein along with knowledge of person of skill in this art provides a catheter having an imaging device that is an Intra-Cardiac Echocardiography Transducer in combination with a pre-formed shape-memory needle and needle sheath, “smart sheath” to facilitate successful transseptal puncture procedures. The pre-formed shape-memory needle and needle sheath deploy within the right atrium by sliding an outer catheter sheath back to expose the needle and needle sheath. Upon release from the outer catheter sheath, the pre-formed shape-memory needle and needle sheath re-establish their pre-programmed three-dimensional conformation which positions the needle and needle sheath in the correct anatomical location for successful transeptal puncture. The imaging device may be any device sufficient to provide imaging.

Also provided is a therapeutic device that is a trans septal puncture device. The therapeutic device can be any applicable therapeutic device.

Also provided herein is a co-axial, dual catheter system in which an outer catheter contains both the imaging device and the second catheter, which itself contains a therapeutic device.

The use of shape-memory materials allow the user who is deploying the apparatus to positively orient the imaging device and the introducer.

Also provided is the ability to manipulate the imaging device and the therapeutic device independently of one another while deployed.

Further, the present invention provides the ability of the imaging device to view the entirety of the procedure in real time.

Accordingly, there is provided in one preferred embodiment, a catheter apparatus for performing intra-cardiac trans-septal puncture with continuous imaging, comprising: an elongated outer catheter body having a proximal end, a distal end, and a peripheral wall defining an outer lumen; disposed within the lumen of the outer catheter body is an imaging transducer apparatus, and an elongated inner catheter body; the inner catheter body has a proximal end, a distal end, and a peripheral wall defining an inner lumen, the distal end of the inner catheter body is a pre-formed shape-memory needle sheath housing a pre-formed shape-memory needle assembly; and, the needle assembly comprises a needle disposed within an introducer.

In further preferred embodiments, there is provided wherein the needle is an RF ablation needle, or wherein the needle is a mechanical needle.

In another preferred embodiment, there is provided wherein the imaging transducer apparatus is an elongated body having a proximal end and a distal end, the imaging transducer is mounted on the distal end, and wherein the imaging transducer apparatus moves freely within the lumen of the outer catheter body and is independent from and not mounted on the inner catheter body.

It is contemplated that the imaging transducer apparatus provides images of the entire procedure in real time.

Also provided herein is a method of creating a trans-septal puncture within the heart of a patient, comprising the steps of:

(a) advancing the catheter apparatus of claim 1 to the right atrium or superior vena cava of the patient;
(b) sufficiently withdrawing the outer catheter body to expose and position the imaging transducer apparatus and the inner catheter body;
(c) imaging the atrial septum and advancing the needle assembly to create a trans-septal puncture. The method of claim 6, further comprising wherein the catheter is introduced percutaneously and enters a heart ventricle through the apex of the heart.

In another preferred embodiment, a method of imaging internal body tissue and accurately puncturing and entering any body cavity or vessel adjacent to a blood vessel by utilizing the catheter apparatus of claim 1.

In another preferred embodiment, the method of the preceding paragraph, further comprising wherein the body cavity is a heart ventricle.

In another preferred embodiment, the method of paragraph 24, further comprising wherein the body cavity is the thoracic cavity or one of its subdivisions.

In another preferred embodiment, the method of paragraph 24, further comprising wherein the body cavity is the abdominopelvic cavity or one of its subdivisions.

### BRIEF DESCRIPTION OF THE DRAWINGS

FIGS. 1A and 1B are graphic drawings showing a preferred embodiment of the present invention in a deployed position (1A) and a retracted position (1B).

FIGS. 2A and 2B are graphic drawings showing another preferred embodiment of the present invention in a deployed position (2A) and a retracted position (2B).

FIG. 3 is a gray-scale radiological image from an echocardiogram and shows a mock simulation of the right atrium that was imaged using Intra Cardiac Echocardiography machine while a trans septal needle catheter was inserted as standard practice.

FIG. 4 is an Artist diagram of anatomy and device in place.

FIG. 5 A-D is a series of radiological images. FIG. 5A is a fluoroscopic image of a transseptal needle. FIG. 5B is an ICE image with left atrium and right atrium marked.

FIG. 5C is a fluoroscopic image in the LAO projection. FIG. 5D is a TOE image with transducer angled to provide a 110 degree bicaval view.

### DETAILED DESCRIPTION OF THE INVENTION

The left atrium (LA) is the most difficult cardiac chamber to access percutaneously. Although it can be reached via the left ventricle and mitral valve, manipulation of catheters that have made two 180° turns is cumbersome. The transseptal puncture permits a direct route to the LA via the intra-atrial septum and systemic venous system. Previously the technique was used infrequently by cardiologists for mitral valvuloplasty and ablation in the left heart; however, the explosion of interest in catheter ablation of atrial fibrillation (AF) has meant the transseptal puncture is a routine skill of the modern cardiac electrophysiologist and interventionalist. (Heart 2009; 95:85-92 doi:10.1136/hrt.2007.135939).

Recently there has been renewed interest in transseptal left heart catheterization caused by the development of left sided radiofrequency catheter ablation. Although conventional left sided accessory pathway ablation is effectively performed with a retrograde approach, severe complications have been reported including damage of the aortic valve, peripheral arterial thromboembolic events, transient ischemic attacks, dissection of the aorta, and endocarditis. Some investigators prefer a transseptal approach towards the mitral ring, which gives improved catheter stability and has a comparable success rate.

Transseptal left heart catheterization has been performed as an alternative to the retrograde approach since 1958. However, a significant number of acute and potentially lethal complications with transseptal puncture may occur. These may occur because of insufficient anatomical landmarks (Gonzalez et al. JACC 2001).

Complications in current method include: (1) Puncture of the wall of the heart; (2) Cardiac tamponade; (3) Systemic emboli; and (4) Aortic perforation.

These complications arise primarily due to the transeptal needle's inadvertent placement due to inadequate information received by the cardiologist. This present inventive device provides the missing information and tools that will make the procedure faster and safer.

This device is contemplated and designed to be used by cardiologists in the Cardiac Catheterization Laboratory, including Interventional cardiologists, and Cardiac Electrophysiologists

This present inventive device is first a “smart material” singular device that uses Intracardiac Electrocardiography (ICE) to continuous image the area within the heart while the transeptic puncture is performed. The smart material will provide the preformed shape that will speed up the procedure as the needle will be quickly deployed upon placement. This device is believed to be novel as in previous experiments in the literature this was done utilizing two catheters (one for ICE and an introducer/needle). By using a single device, it speeds up the procedure, reduces the chances of complication by having more than one catheter in the patient, and makes the procedure easier to perform; hence this device minimizes the potential risks for acute complications in patients undergoing trans septal puncture.

Some of the advantages of the present inventive device include improvements in: (1) Field of View—The operator can decide were the needle is based upon visual view of anatomical landmarks; (2) Can use standard trans septal technique and needle; (3) The imaging guided needle puncture will be safer as location can be clearly identified visually. Due to anatomical differences among patients, it can be difficult to accurately puncture the fossa in some patients. This device would take away doubt concerning the location of the puncture site; (4) The continuous view will allow more certainty of the puncture site and therefore the procedure will be faster.

This catheter is contemplated for use in structural heart disease treatment such as mitral valve replacement (the percutaneous heart valve market size is conservatively estimated to be a US$2 billion). It can also be used in and cardiac electrophysiology (ablations). The global atrial fibrillation market to reach US$4.1 Billion by 2015 according to http://www.StrategyR.com/.

In particular, this device comprises a catheter device with an ultrasonic device mounted on the proximal end of the catheter. The catheter is double sheathed with one sheath having the ultrasonic sensor at the proximal end. The second sheath is the pathway for the introducer and the needle and is a peel away smart material design. Once the catheter is in place, the peel away section detaches from the main body and is positioned under the guidance of the view provided by the images returned from the ultrasonic sensor. The peel away design is believed to be one of the important critical features for the success and novel nature of this design. As the ultrasonic sensor hovers over the peel away section at a distance, it will afford a overhead view of the introducer and needle position. This is critical for success as this will give a big and relevant field of view for the needle driven puncture.

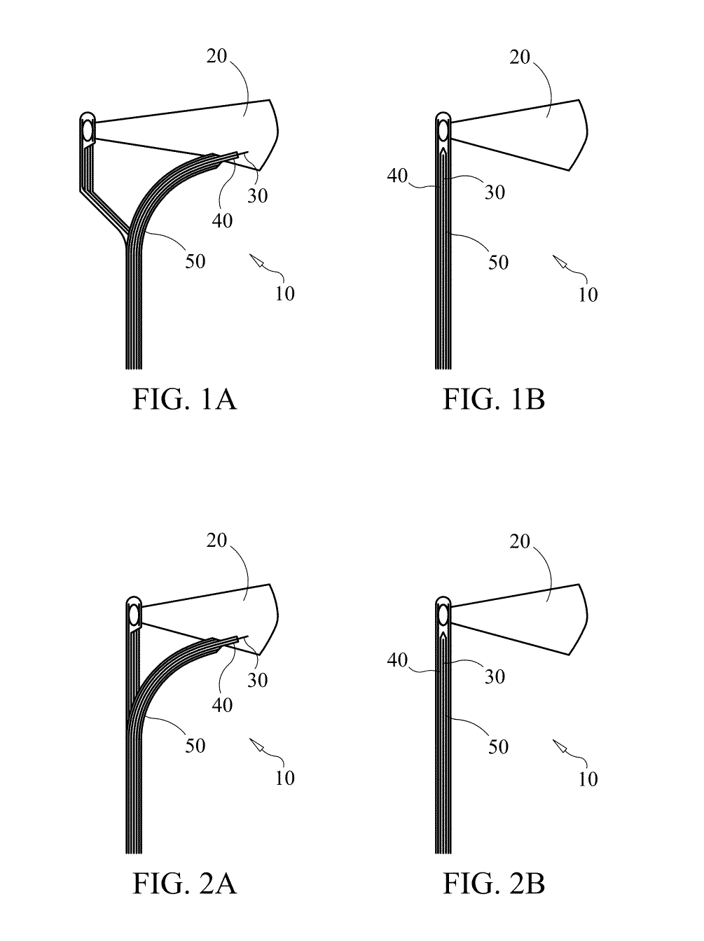

Referring now to the figures, FIG. 1A-B and FIG. 2A-B are graphic drawing sets showing a preferred embodiment of the present invention in a deployed position (1A, 2A) and a retracted position (1B, 2B).

In operation, the singular device (as shown in the illustrations) is used as follows:
A. Advance wire to Superior Vena Cava.
B. Advance needle freeing smart design sheath from catheter.
C. Pullback and find proper puncture site via built in Inter Cardiac Echocardiography.
D. Puncture site. Advance catheter
E. Advance wire into Left Atrium
F. Remove device.

This device will consist of a catheter apparatus comprising:
1. An elongated flexible body having proximal and distal ends to the user. An ultrasonic transducer will be mounted on the distal end of the catheter body to transmit and receive ultrasound energy thus providing a field of view within which anatomical landmarks can be discerned and fluid flow visualized.
2. The catheter body containing the ultrasonic transducer will be formed such that the needle sheath will fit snugly within the shape to enable ease of insertion via the body vessels to the target site (i.e. right atrium). The needle sheath will remain dormant until the operator his ready to deploy it.
3. The needle sheath will have its' shape preprogrammed due to the use of shape memory alloys (smart material such as Nitinol) Once released from the catheter, the needle sheath and needle will take up its' original shape. This will enable the rapid and targeted deployment of the needle hence assuring successful trans septal access.
4. Steerability of the device is by the manipulation of the needle.

An electrical conductor is disposed within the outer catheter body for electrically connecting the transducer to control circuitry external of the catheter. A therapeutic device can be delivered from proximate to the distal end of the outer catheter for operation within the ultrasonic transducer field of view. The therapeutic device moves independently of the transducer, both sharing the lumen of the outer catheter. In some embodiments, the therapeutic device is co-axial with the transducer apparatus. A guide wire is optionally further disposed in the outer catheter body and extends from the proximal end of the catheter body to the distal end of the catheter body.

The present invention further relates to a medical system comprising a shape-memory catheter as described herein, control circuitry means for controlling operation of an ultrasonic transducer disposed on the catheter and display means for displaying flow rates and features imaged by the ultrasonic transducer.

The present invention also relates to a method of therapeutic intervention in a living body. The method includes the steps of inserting a catheter as described herein into the living body. A surgical device is inserted into the body through a port disposed in the catheter body and extending from the proximal end of the catheter body to the distal end of the catheter body. An ultrasonic transducer apparatus disposed within the catheter body is pulsed to transmit ultrasound and receive resultant echoes. The surgical device is operated within a field of view provided by the ultrasonic transducer. The resultant echoes are processed to image the operation of the surgical device.

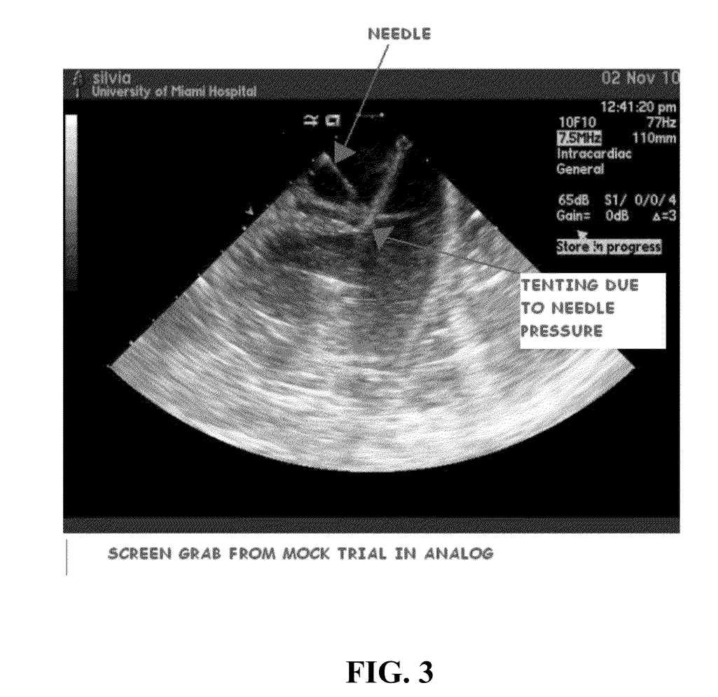

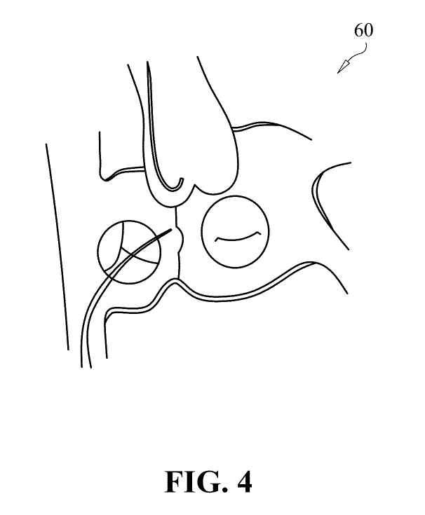

FIG. 3 shows right atrium that was imaged using Intra Cardiac Echocardiography machine while a trans septal needle catheter was inserted as standard practice. FIG. 3 shows needle (red arrow) and shows tenting due to needle pressure (red arrow).

FIG. 4 is an Artist diagram of anatomy and device in place. FIG. 4 shows a left anterior oblique view of a heart and shows a pigtail diagnostic catheter in an aortic route. The mitral and tricuspid valve are seen and the fossa ovalis (target) is illustrated.

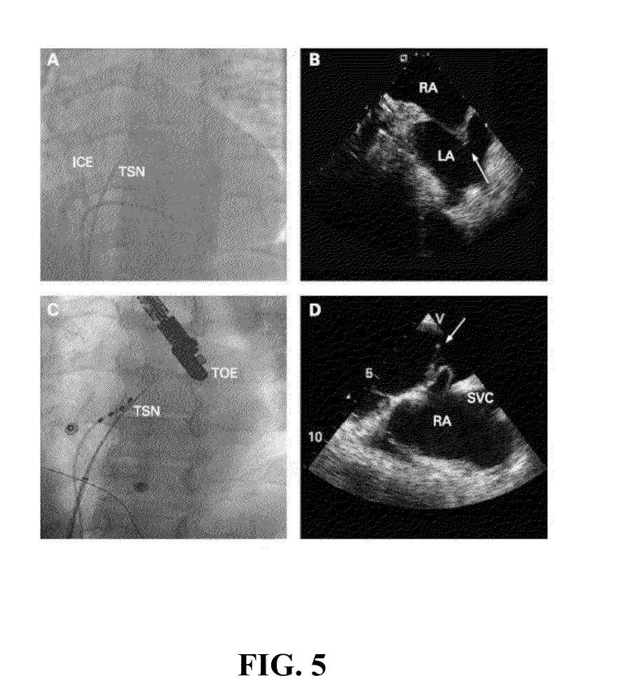

FIG. 5 A-D is a series of radiological images. FIG. 5A is a fluoroscopic image of a transseptal needle abutting the intra-atrial septum in the left anterior oblique (LAO) projection. A phased array transducer for intra-cardiac echocardiography (ICE) is positioned in the right atrium and is turned to face the intra-atrial septum. An additional quadripolar catheter is seen. FIG. 5B is a corresponding ICE image with left atrium and right atrium marked. The white arrow indicates the transseptal needle which is tenting the fossa ovalis and prolapsing it into the LA. FIG. 5C is a fluoroscopic image in the LAO projection inn which the transseptal needle is against the septum and the transesophageal (TOE) probe is in the esophagus. An additional quadripolar catheter is seen. FIG. 5D is a TOE image with transducer angled to provide a 110 degree bicaval view. The white arrow shows the needle tenting the fossa and prolapsing it into the LA, and superior vena cava (SVC).

It is appreciated that the other types of ultrasonic transducers can be used in the present invention, such as any mechanical types, or any dynamic array types, or any offset stereoscopic imaging types, or any multidimensional imaging types incorporated into a virtual reality environment so that all forms of field of views, such as 1) tomographic (slices), 2) stereoscopic, 3) three-dimensional, 4) virtual reality (multidimensional) can be provided in the present invention. In preferred embodiments, the transducer is miniaturized to facilitate co-location with the needle and needle-sheath within the lumen of the outer catheter.

In the preferred embodiment of the present invention, the ultrasonic transducer preferably has a frequency of 5 to 20 megahertz (MHz) and more preferably a frequency of 7 to 10 MHz. Intracardiac imaging in an adult is believed to require image penetration of up to 2 to 10 centimeters (cm). Transducer circuitry including signal transduction and transmission logic and software, amplification and filtering logic and software, imaging logic and software, and interpretation and analytic logic and software are all conventional and included within the scope of the present invention are transducer devices publicly known as of the effective date of the present invention. An example of a preferred phased array transducer and the accompanying circuitry and the imaging console may be obtained from Endosonics of Rancho Cordova, Calif., or Intravascular Research Limited (United Kingdom).

In the preferred embodiment, the outer catheter body preferably has a diameter ranging from about 4 to about 24 French [one French divided by Pi equals one millimeter (mm)] and, more preferably a diameter of 6 to 12 French. However, these details are within the level of skill of a person versed in this art.

The inner catheter body (needle sheath) and needlemay be fabricated from a variety of materials including, but not limited to, stainless steel, Nitinol™, platinum and polymers, so long as they maintain the functionality of being a pre-formable shape-memory material.

Finally, the catheter apparatus described herein above comprises a versatile tool capable of reaching any point within the human body serviced by a blood vessel of sufficient diameter. Thus, the device can be used to provide a pathway to body cavities and vessels in addition to the heart atria, including but not limited to heart ventricles, the thoracic cavity and its subdivisions and organs, or the abdonimopelvic cavity and its subdivisions and organs. The device as described is therefore capable of reaching tissue in any such area, imaging such tissue, puncturing such tissue, and accordingly, moving from vessel to cavity throughout the body.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed as the invention:

1. A catheter apparatus for performing intra-cardiac trans-septal puncture with continuous imaging, comprising:
an elongated outer catheter body having a proximal end, a distal end, and a peripheral wall defining an outer lumen;
disposed within the lumen of the outer catheter body is an imaging transducer apparatus, and an elongated inner catheter body;
the inner catheter body has a proximal end, a distal end, and a peripheral wall defining an inner lumen, the distal end of the inner catheter body is a pre-formed shape-memory needle sheath housing a pre-formed shape-memory needle assembly; and
the needle assembly comprises a needle disposed within an introducer.

2. The catheter of claim 1, wherein the needle is an RF ablation needle.

3. The catheter of claim 1, wherein the needle is a mechanical needle.

4. The catheter of claim 1, wherein the imaging transducer apparatus is an elongated body having a proximal end and a distal end, the imaging transducer is mounted on the distal end, and wherein the imaging transducer apparatus moves freely within the lumen of the outer catheter body and is independent from and not mounted on the inner catheter body.

5. The catheter of claim 1, wherein the imaging transducer apparatus provides images of the entire procedure in real time.

6. A method of creating a trans-septal puncture within the heart of a patient, comprising the steps of:
(a) advancing the catheter apparatus of claim 1 to the right atrium or superior vena cava of the patient;
(b) sufficiently withdrawing the outer catheter body to expose and position the imaging transducer apparatus and the inner catheter body; and
(c) imaging the atrial septum and advancing the needle assembly to create a trans-septal puncture.

7. The method of claim 6, further comprising wherein the catheter is introduced percutaneously and enters a heart ventricle through the apex of the heart.

8. A method of imaging internal body tissue and accurately puncturing and entering any body cavity or vessel adjacent to a blood vessel by utilizing the catheter apparatus of claim 1.

9. The method of claim 8, further comprising wherein the body cavity is a heart ventricle.

10. The method of claim 8, further comprising wherein the body cavity is the thoracic cavity or one of its subdivisions.

11. The method of claim 8, further comprising wherein the body cavity is the abdominopelvic cavity or one of its subdivisions.
